(19) United States
(12) Patent Application Publication
(10) Pub. No.: US 2025/0296964 A1
(43) Pub. Date: Sep. 25, 2025

(54) USE OF A 53BP1 BINDING HUMAN UBIQUITIN VARIANT TO IMPROVE RATES OF HDR IN MULTIPLE CELL TYPES

(71) Applicant: Integrated DNA Technologies, Inc., Coralville, IA (US)

(72) Inventors: Steve Ehren Glenn, North Liberty, IA (US); Bernice Thommandru, Iowa City, IA (US); Karthik Murugan, Coralville, IA (US); Christopher Anthony Vakulskas, North Liberty, IA (US)

(21) Appl. No.: 18/613,719

(22) Filed: Mar. 22, 2024

(51) Int. Cl. C07K 14/435; C12N 9/22; C12N 15/90

(52) U.S. Cl. CPC C07K 14/435; C12N 9/22; C12N 15/902; C12N 2310/20

(57) ABSTRACT

The present invention pertains to methods of improving homology directed repair in a recipient cell. The method includes a step of expressing a Ubiquitin polypeptide variant corresponding to SEQ ID NO:1 in the recipient cell. An improvement of homology directed repair in the recipient cell is increased as compared a cell that is not treated with the Ubiquitin polypeptide variant. Methods of improving CRISPR ribonucleoprotein complex-mediated gene editing using the Ubiquitin polypeptide variant corresponding to SEQ ID NO:1 as an enhancer of homology directed repair are also provided.

### SEQUENCE LISTING

The instant application contains a Sequence Listing which has been submitted in XML format via Patent Center and is hereby incorporated by reference in its entirety. Said XML copy, created on Mar. 15, 2023, is named IDT01-024-PRO.xml, and is 33,751 bytes in size.

### FIELD OF THE INVENTION

This invention pertains to the ability of a ubiquitin variant to bind to 53BP1 and bias the repair of a double-strand break (DSB) towards homology directed repair (HDR).

### BACKGROUND OF THE INVENTION

Double-strand breaks (DSBs) are predominantly repaired through two mechanisms, non-homologous end joining (NHEJ), in which broken ends are rejoined, often imprecisely, or homology directed repair (HDR), which typically involves a sister chromatid or homologous chromosome being used as a repair template. HDR is facilitated by the presence of a sister chromatid and there are cellular mechanisms in place biasing repair towards NHEJ during the G1 phase of the cell cycle¹. A key determinant of repair pathway choice is 53BP1. 53BP1 was first described as a binding partner of the tumor suppressor gene p53 and was later shown to be a key protein in NHEJ ². 53BP1 rapidly accumulates at sites of double strand breaks. In G1, 53BP1 recruits RIF1 and inhibits end resection ^{3,4}. End resection is a critical step in repair pathway choice, as it is necessary for HDR and inhibits NHEJ ¹. By inhibiting end resection, 53BP1 biases repair towards NEHJ and consequently loss of 53BP1 results in increased HDR 5. Targeted nucleases can be introduced into cells in conjunction with a DNA repair template with homology to a targeted cut site to facilitate precise genome editing via HDR ⁶. A strong inhibitor of 53BP1 is therefore useful for precise genome editing.

The recruitment of 53BP1 to DSB sites is dependent upon both H4K20 methylation and H2AK15 ubiquitination. 53BP1 has tandem Tudor domains that have been shown to specifically bind mono and demethylated H4K20 and H4K20 methylation was shown to be important for 53BP1 recruitment to double strand breaks ^{7,8}. Introducing D1521R, a mutation that disrupts the activity of the Tudor domain, impairs the ability of 53BP1 to form ionizing radiation-induced foci ⁹. The minimal focus-forming region of 53BP1 consists of the Tudor domain flanked by an N-terminal oligomerization region and a C-terminal extension. Notably, 53BP1 accumulation at DSBs requires the E3 ubiquitin ligase RNF168, that mediates H2AK13 and H2AK15 ubiquitination¹⁰. The C-terminal extension was shown to contain a ubiquitination-dependent recruitment motif (UDR) that binds specifically to H2AK15ub and is required for 53BP1 recruitment to DSB sites ⁹.

Due to the affinity of 53BP1 for ubiquitinated H2A, a screen of ubiquitin variants for interaction with 53BP1 was conducted recently by Canny et al. in which they discovered and modified a ubiquitin variant with selective binding to 53BP1 that they named i53 (inhibitor of 53BP1) ¹¹. The top five hits from the ubiquitin variant screen were A10, A11, C08, G08, and H04, with G08 having the highest affinity. In contrast to what might be expected, the interaction of 53BP1 with G08 did not require the UDR and the interaction was shown to be between G08and the 53BP1 Tudor domain. To generate i53, G08 was modified by introducing an I44A mutation that disrupts a solvent exposed hydrophobic patch on ubiquitin that most ubiquitin binding proteins interact with ^{9, 12}. Notably, this mutation in the context of H2AK_{c}15ub(I44A) interferes with 53BP1 interaction with ubiquitinated H2A, yet does not interfere with the ability of i53 to enhance HDR, consistent with i53 enhancing HDR through interaction with the 53BP1 Tudor domain and not the UDR domain ^{9, 11}. Additionally, i53 was modified relative to G08 through the removal of the C-terminal di-glycine motif. Introduction of i53, but not a 53BP1 binding deficient i53 variant DM (i53 P69L+L70V), into cells inhibited the formation ionizing radiation induced 53BP1 foci. Introduction of i53 via plasmid delivery, adeno-associated virus mediated gene delivery, or delivery of mRNA were all shown to improve the rates of HDR. Rates of HDR were improved with the introduction of i53 using both double-stranded DNA donors and using single-stranded DNA donors, which have been shown to use different HDR mechanisms ^{11, 13, 14}.

Our previous disclosures described a screen to identify ubiquitin variants (Ubvs) with increased affinity for 53BP1 and improved efficacy for enhancing HDR rates. In that screen we interrogated the effect of all possible single amino acid substitutions individually at every position in i53 (a.a. 1-74) on the expression of a reporter gene in a two-hybrid assay in E. coli. From that screening method, 240 amino acid changes were identified as candidates for improving the affinity of i53 for 53BP1. When mutations were combined together, the highest affinity Ubv (CM1) had a 50-fold to 100-fold improvement in the affinity for a fragment of 53BP1 relative to i53. We demonstrated that Ubv-A was able to improve rates of HDR in HEK293 using ssDNA donor, dsDNA donor, and AAV donor and that we were also able to boost HDR in K562 cells with AAV donor.

While expression of 53BP1 has low tissue specificity being broadly expressed throughout all human tissues, the degree to which 53BP1 represses HDR in different cell types is unclear. We hypothesize that inhibition of 53BP1 using a ubiquitin variant may be beneficial to HDR in any human cell type that has detectible levels of HDR. Further, any ubiquitin variant engineered to bind human 53BP1, such as our CM1 protein, would likely also be beneficial for HDR in cells from any species where there is strong conservation in the tudor domain of 53BP1 between the human 53BP1 protein and the 53BP1 protein from that species, for example the region of human 53BP1 used for the crystal structure of i53 and 53BP1 (a.a. 1484 to 1603) is 100% conserved at the amino acid level between humans, mice, and pigs. It is therefore likely that CM1 or our other improved affinity ubiquitin variants would be effective in boosting HDR in cells from those animals.

### BRIEF SUMMARY OF THE INVENTION

In a first aspect, a method of improving homology directed repair in a recipient cell is provided. The method includes a step of expressing a Ubiquitin polypeptide variant corresponding to SEQ ID NO:1 in the recipient cell. An improvement of homology directed repair in the recipient cell is increased as compared to a cell that is not treated with the Ubiquitin polypeptide variant.

In a second aspect, a method of improving CRISPR-mediated gene editing in a target cell is provided. The method includes the step of transfecting a Ubiquitin polypeptide variant corresponding to SEQ ID NO:1 or a Ubiquitin mRNA variant corresponding to SEQ ID NO:31 into the target cell that harbors a CRISPR ribonucleoprotein complex.

### BRIEF DESCRIPTION OF THE DRAWINGS

FIG. 1A depicts an exemplary comparison of fold increase in HDR in HEK293 and Jurkat cells. Panel 1: The graph shows the percent HDR measured by either NGS (HEK293 cells, blue bars) or by EcoR1 digestion (Jurkat cells, red bars), for cells that had either no Ubv-A (lighter bars) or Ubv-A of the indicated dose (darker bars) co-delivered with Cas9 RNP and donor Panel 2: The graph shows the fold improvement in HDR across multiple sites in both HEK293 (blue) and Jurkat (red) cells. Error bars indicate standard deviation from at least two replicates.

FIG. 1B depicts a graph showing the percent perfect HDR for editing with and without Ubv-A protein or mRNA in primary T Cells from two donors. Cas9 RNP (4 μM), ssDNA donor (3 μM), Cas9 electroporation enhancer (3 μM), along with either no Ubv-A, 25 μM Ubv-A protein, or 13 nM Ubv-A mRNA was delivered into primary T cells by Lonza nucleofection (pulse code EH-115), genomic DNA was isolated after 48 hours and analyzed by NGS.

FIG. 1C depicts a graph showing the percent perfect HDR for editing with and without Ubv-A in iPSCs. Cas9 RNP (4 μM), ssDNA donor (2 μM), and either 0 or 25 μM Ubv-A was delivered into iPSCs by Lonza nucleofection, genomic DNA was isolated after 96 or 120 hours and analyzed by NGS.

### DETAILED DESCRIPTION OF THE INVENTION

The goal of this work is to test how broadly effective our ubiquitin variants are in boosting HDR in different human cell types. To do so we tested the ability to improve HDR in Jurkat cells (an immortalized line of human T lymphocytes), primary human T cells, and induced pluripotent stem cells (iPSCs). This invention disclosure pertains to testing the ability of CM1 (hereafter referred to as ubiquitin variant A [Ubv-A] (SEQ ID NO:1), see Table 1) to improve rates of HDR in these various cell types.

TABLE 1
Amino acid and DNA sequences

| | Protein Sequence | DNA sequence |
|---|---|---|
| Name | (SEQ ID NO:1) | (SEQ ID NO: 2) |
| Ubv-A | MLIFVRMLTGKMIELEVEPSDTIE | ATGTTGATTTTCGTACGCATGTTGACTG |
| (previously | NVKAKIQDHEGIPPDQQRLAFQGK | GAAAGATGATCGAGTTGGAAGTGGAGCC |
| called CM1) | SLEDGRTLSDYNILKDPKKMPLLR | TTCCGATACTATCGAGAATGTTAAGGCC |
| | LR | AAAATCCAAGATCATGAAGGGATTCCTC |
| | | CAGATCAACAACGCCTTGCTTTTCAAGG |
| | | GAAGAGCCTGGAGGACGGTCGCACACTG |
| | | TCTGACTATAACATTCTTAAAGATCCTA |
| | | AAAAGATGCCACTGCTGCGCTTGCGT |

For all experiments, genome editing was mediated via IDT Alt-R Cas9 ribonucleoprotein (RNP) complexes delivered by Lonza nucleofection in concert with single-stranded oligodeoxynucleotide (ssODN) HDR repair templates. The specific repair event was the insertion of either a 6-nt EcoRI sequence (5′-GAATTC-3′) or a 33-nt sequence encoding an HA tag directly at the canonical Sp Cas9 cut site (between bases 3 and 4 in the 5′-direction from the PAM sequence) with the exception of editing at HBB which involved multiple small edits. RNP complexes were formed with a nuclease-specific guide for the SERPINCl, HPRTl, MET, CD5, or HBB genes. The sgRNAs corresponding to the protospacer sequences listed in Table 2 were ordered from IDT as Alt-R sgRNAs (Table 2). HDR donor templates contain 40-nt homology arms (HA) on the 5′ and 3′ ends with the exception of the HBB donor that used longer HAs. The HDR donor sequences listed in Table 3 were ordered from IDT as Alt-R HDR donor oligos (Table 3).

TABLE 2
sgRNA designs

| Name | |
|---|---|
| (SEQ ID NO:) | Protospacer Sequences |
| CD5 sgRNA | CAGTCGCTTCCTGCCTCGGA |
| (SEQ ID NO: 3) | |
| HPRT1 sgRNA | AATTATGGGGATTACTAGGA |
| (SEQ ID NO: 4) | |
| MET sgRNA | CAAAGTCCTTTCATCTGTAA |
| (SEQ ID NO: 5) | |
| SERPINC1 sgRNA | ACCTCTGGAAAAAGGTAAGA |
| (SEQ ID NO: 6) | |
| HBB sgRNA | CTTGCCCCACAGGGCAGTAA |
| (SEQ ID NO: 7) | |

TABLE 3
Donor DNA sequences

| Name | |
|---|---|
| (SEQ ID NO:) | Sequence |
| CD5 Donor | ACCCTCCTCTCTTCTTTCTGCAGTCGCTTCCTGCCTCGGATACCCA |
| (SEQ ID NO: 8) | TACGATGTGCCTGATTACGCAGGATCACGGCTCAGCTGGTATGAC |
| | CCAGGTAAGGAAGAGCCACATG |
| HPRT1 Donor | AAAGACTATGAAATGGAGAGCTAAATTATGGGGATTACTAGAATTC |
| (SEQ ID NO: 9) | GGAAGGGGCAGCAATGAGTTGACACTACAGACAAGGCACT |
| MET Donor | TGTGTGGTGAGCGCCCTGGGAGCCAAAGTCCTTTCATCTGGAATTCT |
| (SEQ ID NO: 10) | AAAGGACCGGTTCATCAACTTCTTTGTAGGCAATACCAT |
| SERPINC1 Donor | ATTCCAATGTGATAGGAACTGTAACCTCTGGAAAAAGGTAGAATTCAG |
| (SEQ ID NO: 11) | AGGGGTGAGCTTTCCCCTTGCCTGCCCCTACTGGGTTT |
| HBB Donor | CAGAGCCATCTATTGCTTACATTTGCTTCTGACACAACTGTGTTC |
| (SEQ ID NO: 12) | ACTAGCAACCTCAAACAGACACCATGGTGCACCTGACTCCTGTGG |
| | AAAAATCGAATTCCGCAGTCACTGCCCTGTGGGGCAAGGTGAACG |
| | TGGATGAAGTTGGTGGTGAGGCCCTGGGCAGGTTGGTATCAAGGTT |
| | ACAAGACAGGTTTAAGGAG |

The 86-nt or 113-nt repair template was homologous to the non-targeting strand of dsDNA, where targeting/non-targeting is defined with respect to the guide RNA sequence and the presence of the NGG PAM sequence identifying the non-target strand. The RNPs were generated by complexing IDT Alt-R Cas9 to IDT Alt-R sgRNA at a 1:1.2 ratio of protein to guide to give the indicated final concentration for each figure where the final concentration of Cas9 RNP refers to the concentration of Cas9 in the final cells, protein, RNA, and DNA mix. The Ubv protein was added to the Cas9 RNP at varying amounts (depending on the experiment) along with donor DNA at the indicated concentrations. Cas9 RNP, donor, and Ubv protein was delivered into cells using the Lonza 96-well Shuttle with the pulse code EH-115 for T-cells, CL-120 for Jurkat cells, or CA-137 for iPSCs. Measurement of HDR by EcoR1 digest was performed by amplification of the region around the Cas9 target cut site using the primers listed in Table 4, followed by digestion with EcoR1 and analysis of the percent cut vs uncut product using an Agilent Fragment Analyzer to calculate percent HDR.

TABLE 4
Primer sequences

| | | | DNA sequence |
|---|---|---|---|
| Gene | Assay | Primer | (SEQ ID NO:) |
| HPRT1 | PCR + EcoR1 digest | Forward | AAGAATGTTGTGATAAAAGGTGATGCT |
| | | | (SEQ ID NO: 13) |
| | | Reverse | ACACATCCATGGGACTTCTGCCTC |
| | | | (SEQ ID NO: 14) |
| MET | PCR + EcoR1 digest | Forward | CCATACCATTCCTCTCCACACAA |
| | | | (SEQ ID NO: 15) |
| | | Reverse | GGGAACTGATGTGACTTACCCTATT |
| | | | (SEQ ID NO: 16) |
| SERPINC1 | PCR + EcoR1 digest | Forward | CTTGTCCCTCTTTGCCTTCTCT |
| | | | (SEQ ID NO: 17) |
| | | Reverse | GGGTGGATCTGAGTGGAAGAAA |
| | | | (SEQ ID NO: 18) |
| HBB | PCR + EcoR1 digest | Forward | AGATATATCTTAGAGGGAGGGCTGA |
| | | | (SEQ ID NO: 19) |
| | | Reverse | GCAGAGATATTGCTATTGCCTTAAC |
| | | | (SEQ ID NO: 20) |
| HPRT1 | NGS (HEK293 cells) | Forward | ACACTCTTTCCCTACACGACGCTCTTCCGATCT |
| | | | TCAGAACTGTCCTTCAGGTTCrCAGGTC/3SpC3/ |
| | | | (SEQ ID NO: 21) |
| | | Reverse | GTGACTGGAGTTCAGACGTGTGCTCTTCCGATC |
| | | | TCAGGGCATTACAAAAGAGAAGArCTGACC/3SpC3/ |
| | | | (SEQ ID NO: 22) |
| MET | NGS | Forward | ACACTCTTTCCCTACACGACGCTCTTCCGATCT |
| | | | CTGCATATTCTCCCCACAGATArGAAGAC/3SpC3/ |
| | | | (SEQ ID NO: 23) |
| | | Reverse | GTGACTGGAGTTCAGACGTGTGCTCTTCCGATCT |
| | | | CCTTTAGCCTTCTCACTGATATCrGAATGG/3SpC3/ |
| | | | (SEQ ID NO: 24) |
| SERPINC1 | NGS | Forward | ACACTCTTTCCCTACACGACGCTCTTCCGATCTC |
| | | | TGGTCTTCTCAAAGGTGTTGrGAGGTG/3SpC3/ |
| | | | (SEQ ID NO: 25) |
| | | Reverse | GTGACTGGAGTTCAGACGTGTGCTCTTCCGATC |
| | | | TTTGCCTCAGATCACACTATCTrCCACTA/3SpC3/ |
| | | | (SEQ ID NO: 26) |
| HBB | NGS | Forward | ACACTCTTTCCCTACACGACGCTCTTCCGATCTCAT |
| | | | GCCCAGTTTCTATTGGT |
| | | | (SEQ ID NO: 27) |
| | | Reverse | GTGACTGGAGTTCAGACGTGTGCTCTTCCGATCTCT |
| | | | AGCAACCTCAAACAGACA |
| | | | (SEQ ID NO: 28) |
| HPRT1 | NGS | Forward | ACACTCTTTCCCTACACGACGCTCTTCCGATCTTCA |
| | (primary T cells) | | GAACTGTCCTTCAGGTTC |
| | | | (SEQ ID NO: 29) |
| | | Reverse | GTGACTGGAGTTCAGACGTGTGCTCTTCCGATCTCA |
| | | | GGGCATTACAAAAGAGAAGA |
| | | | (SEQ ID NO: 30) |

Measurement of HDR by NGS was performed according to the rhAmpSeq CRISPR library preparation protocol available on IDT's website use the primers listed in Table 4 for PCR 1 and standard i5 and i7 indexing primers for PCR2. NGS libraries were then sequenced on a MiSeq® platform (Illumina®) using 2×150 bp PE sequencing and processed through an internal IDT processing pipeline (CISPAltRations). For mRNA experiments, Ubv-A mRNA was produced by in vitro transcription (Table 5).

TABLE 5
mRNA

| Name | mRNA sequence (SEQ ID NO: 31) |
|---|---|
| Ubv-A mRNA | GGGAGACCCAAGCUGGCUAGCGUUUAAACGGGCCCUCUAGACUCGA |
| | GCGGCCGCCACCAUGCUGAUCUUCGUGAGAAUGCUGACCGGCAAGA |
| | UGAUCGAACUGGAAGUGGAACCCAGCGACACCAUCGAGAACGUGAA |
| | GGCCAAAAUCCAGGACCACGAGGGCAUCCCUCCUGACCAGCAGAGA |
| | CUGGCCUUUCAGGGAAAGUCCCUGGAAGAUGGAAGAACCCUGAGCG |
| | ACUACAACAUCCUGAAGGACCCUAAGAAGAUGCCACUGCUGAGACU |
| | GAGAUGAUCAGCCUCGACUGUGCCUUCUAAAAAAAAAAAAAAAAAA |
| | AAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAA |
| | AAAAAAAAAAA |

For T cells, RNPs and HDR donors were delivered using the Lonza Nucleofection system. Single guide RNAs targeting HsHPRT and HsCD5 were hydrated to 100 μM in IDTE pH 7.5. HDR donors were designed with 40 nucleotide homology arms flanking the Cas9 cleavage site. For HPRT a 6nt sequence (GAATTC), and for CD5 a 33nt sequence (HA tag) were designed to be inserted directly into the Cas9 cleavage site. HDR donors were ordered as Alt-R HDR DNA Oligos and hydrated to 100 μM in IDTE pH 7.5. RNPs were generated by complexation of 120 pmol Cas9 V3 protein with 144 pmol sgRNA in a total volume of 5 μL with 1× phosphate-buffered saline (PBS) to adjust to the final volume. Frozen human primary pan-T cells (Stemcell Technologies) from 2 unique human donors were thawed in ImmunoCult-XF T Cell Expansion Medium including 100 ng/ml IL-2, ACF, and activated with 25 uL/mL ImmunoCult CD3/CD28 TCell Activator for 48 hours. Following activation, cells were counted and pelleted using centrifugation (300× g, 10 minutes at room temperature) and washed with 10 mL PBS. Cells were again pelleted and resuspended in Nucleofection Solution P3 at 5e7 cells/mL. For each electroporation, 5 μL of RNP complex was added to 20 μL of cells in P3 (1e6 cells/nucleofection) for a final concentration of 4 uM RNP, HDR Donors and IDT Alt-R Cas9 Electroporation Enhancer were added to achieve a final concentration of 3 μM each, and UbvA peptide at a final concentration of 25 uM in a final nucleofection reaction volume of 30 μL. The solution was mixed by pipetting and 25 μL was transferred to an electroporation cuvette plate. The cells were electroporated according to the manufacturer's protocol using the Amaxa 96-well Shuttle and nucleofection protocol 96-EH-115. After electroporation, the cells were resuspended in 75 μL pre-warmed IL-2 culture media in the electroporation cuvette. Triplicate aliquots of 25 μL of recovered cells were further cultured in 175 μL pre-warmed IL-2 media with and without Alt-R HDR Enhancer V2 at 1uM. The cells were allowed to grow for 72 hours in total, after which genomic DNA was isolated. Perfect HDR was quantified by NGS amplicon sequencing on the Illumina MiSeq platform and data analysis done via IDT's in-house data analysis pipeline (CRISPAltRations).

For iPSCs, PIGI consented human iPSCs, GM23338 from Coriell Institute (NJ, USA) were used. iPSCs were cultured on vitronectin (Catalog #100-0763, Stemcell Technologies, BC, Canada) coated 6-well plates using complete mTeSR Plus medium (Catalog #100-0276, Stemcell Technologies) at 37° C., and 5% CO2 in a humidified incubator. iPSCs were cultured in a 6-well plate for 4 to 5 days to reach a confluence of 90%. Cells were detached using ReLeSR (Catalog #100-0484, Stemcell Technologies) following the manufacturer's protocol. iPSCs were washed in PBS and pelleted by centrifuging at 300× g for 5 minutes. Cells were broken into single cells by gentle pipetting and counted using Countess Automated Cell Counter (Thermo Scientific, Waltham, MA, USA). iPSCs were resuspended in 20 μL P3 buffer (Catalog #:V4XP-3012) at 2×10⁵ cells per nucleofection well. Cas9 RNP, donor, and Ubv protein were added at concentration described above to the resuspended cells to a 25 μL volume and 20 μL was transferred to a 96-well plate 4-D Nucleofector system (Catalog #: AAF-1003S, Lonza) for nucleofection using code CA-137. After electroporation, cells were recovered in complete mTeSR Plus medium with 1X CloneR 2 supplement (Catalog #100-0691, Stemcell Technologies). 25 μL of recovered cells were plated in vitronectin coated 96-well plates with 175 μL complete mTeSR Plus medium with 1X CloneR 2 supplement. After 24 h, the medium was changed to complete mTeSR Plus medium, and the cells were maintained for 4 to 5 days with regular media change until cells were confluent for gDNA extraction. Cells were washed with PBS before gDNA was extracted using 50 μL QuickExtract per well (CAT #QE09050,Lucigen) and following manufacturer's protocol. rhAmpSeq CRISPR Library preparation and subsequent sequencing on the Illumina MiSeq platform was then performed. Data was analyze using IDT's rhAmpSeq Analysis Tool.

### Applications

In a first aspect, a method of improving homology directed repair in a recipient cell is provided. The method includes a step of expressing a Ubiquitin polypeptide variant of SEQ ID NO:1 in the recipient cell. An improvement of homology directed repair in the recipient cell is increased as compared to a cell that is not treated with the Ubiquitin polypeptide variant. In a first respect, the recipient cell is selected from an immortalized cell. In an elaboration of the first respect, the immortalized cell is a HEK293 kidney cell or a Jurkat T cell. In a second respect, the recipient cell is a primary human T cell or an induced pluripotent stem cell. In a third respect, the step of expressing the Ubiquitin polypeptide variant of SEQ ID NO:1 comprises transfecting SEQ ID NO:1 into the recipient cell. In a fourth respect, the step of expressing the Ubiquitin polypeptide variant of SEQ ID NO:1 comprises transfecting SEQ ID NO:31 into the recipient cell, following by translation of SEQ ID NO:31 to produce SEQ ID NO:1.

In a second aspect, a method of improving CRISPR-mediated gene editing in a target cell is provided. The method includes the step of transfecting a Ubiquitin polypeptide variant corresponding to SEQ ID NO:1 or a Ubiquitin mRNA variant corresponding to SEQ ID NO:31 into the target cell that harbors a CRISPR ribonucleoprotein complex. In a first respect, the CRISPR ribonucleoprotein complex includes a Cas9 polypeptide and a suitable guide RNA. In a second respect, the method includes the step of transfecting a Ubiquitin polypeptide variant corresponding to SEQ ID NO:1 into the target cell that harbors a CRISPR ribonucleoprotein complex. In a third respect, the method includes the step of transfecting a Ubiquitin mRNA variant corresponding to SEQ ID NO:31 into the target cell that harbors a CRISPR ribonucleoprotein complex.

### EXAMPLES

### Example 1. Ubv-A is Capable of Boosting HDR in Multiple cell Types

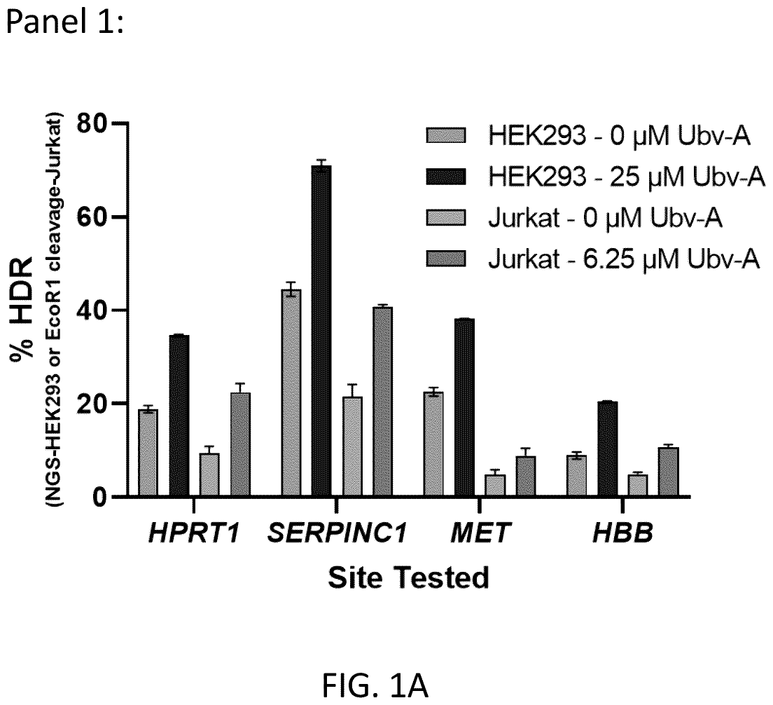

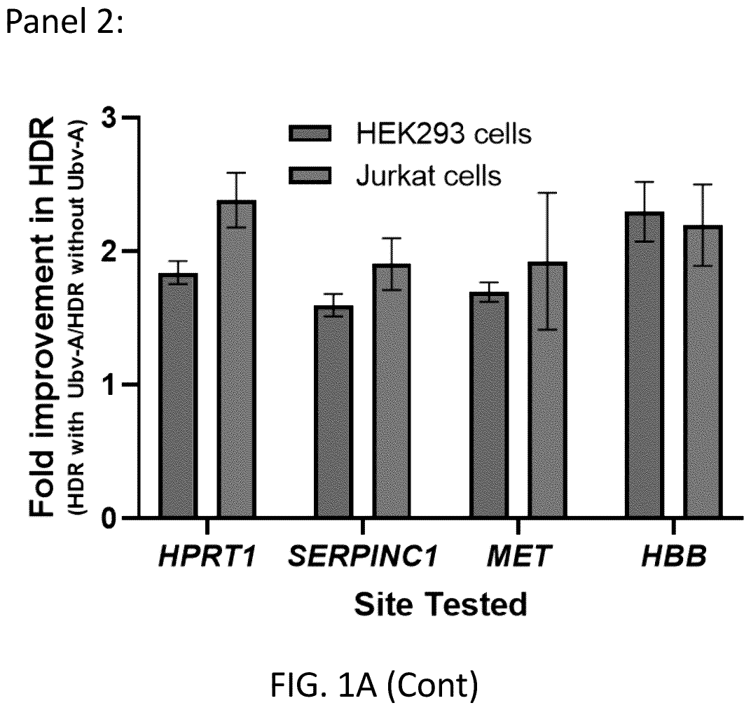

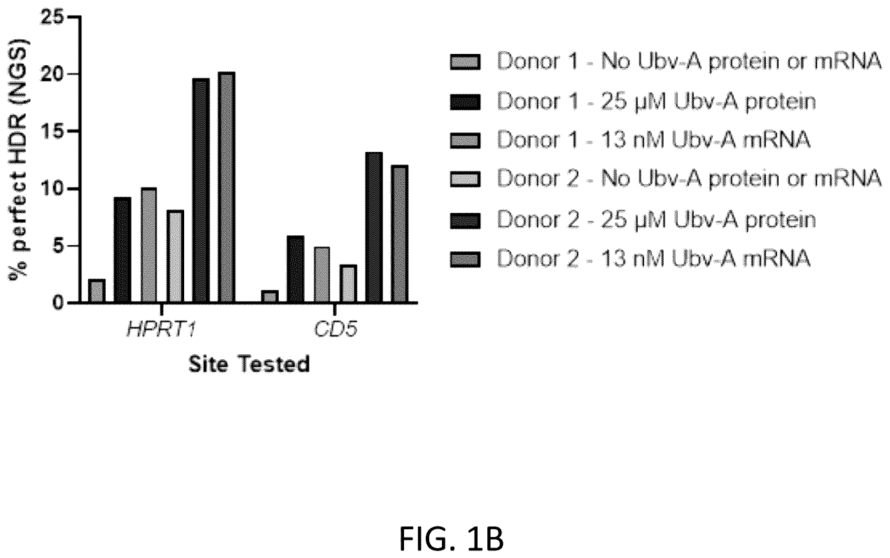

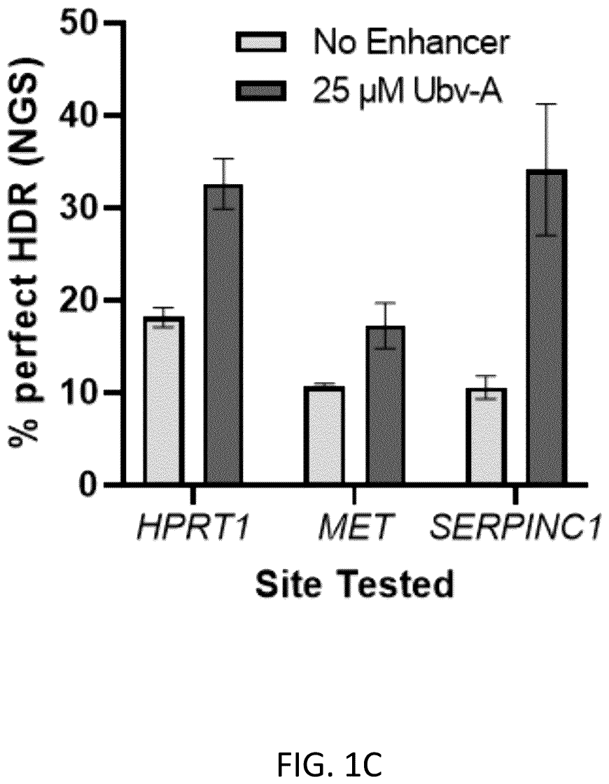

In order to determine if Ubv-A is effective in boost HDR in additional cell types, the effectiveness in both Jurkat and HEK293 cells was compared. Percent perfect HDR was measured by Next-Generation Sequencing (NGS) in HEK293 cells and the % HDR in Jurkat cells was measured by EcoR1 cleavage. The results are shown in FIG. 1A. While the level of HDR varied from site to site and from cell type to cell type, the overall fold improvement in HDR was typically in the range of 1.5-fold to 2-fold, indicating Ubv-A is equally effective in boosting HDR in both HEK293 cells and Jurkat cells. To test whether Ubv-A is also effective in primary T cells Ubv-A protein or mRNA was delivered alongside Cas9 RNP. The results are shown in FIG. 1B. Ubv-A was able to increase rates of HDR by over two-fold. To explore whether Ubv-A is able to boost HDR in induced pluripotent stem cells Ubv-A purified protein was co-delivered with Cas9 RNP into iPSCs. The results are shown in FIG. 1C. While the benefit from Ubv-A was more variable by site in iPSCs, an increase of 1.5-fold to 3-fold was observed in iPSCs. These data indicate that Ubv-A is broadly effective in multiple cell types including HEK293 cells, primary T cells, and iPSCs.

### REFERENCES

1. Chapman, J. R., Taylor, M. R. & Boulton, S. J. Playing the end game: DNA double-strand break repair pathway choice. Molecular Cell 47, 497-510 (2012).
2. Iwabuchi, K., Bartel, P. L., Li, B., Marraccino, R. & Fields, S. Two cellular proteins that bind to wild-type but not mutant p53. PNAS 91, 6098-6102 (1994).
3. Escribano-Díaz, C. et al. A cell cycle-dependent regulatory circuit composed of 53BP1-RIF1 and BRCA1-CtIP controls DNA repair pathway choice. Molecular Cell 49, 872-883 (2013).
4. Feng, L., Fong, K. W., Wang, J., Wang, W. & Chen, J. RIF1 counteracts BRCA1-mediated end resection during DNA repair. J. Biol. Chem. 288, 11135-11143 (2013).
5. Xie, A. et al. Distinct roles of chromatin-associated proteins MDC1 and 53BP1 in mammalian double-strand break repair. Molecular Cell 28, 1045-1057 (2007).
6. Gaj, T., Sirk, S. J., Shui, S. L. & Liu, J. Genome-Editing Technologies: Principles and Applications. Cold Spring Harbor perspectives in biology 8 (2016).
7. Botuyan, M. V. et al. Structural basis for the methylation state-specific recognition of histone H4-K20 by 53BP1 and Crb2 in DNA repair. Cell 127, 1361-1373 (2006).
8. Charier, G. et al. The Tudor tandem of 53BP1: a new structural motif involved in DNA and RG-rich peptide binding. Structure (London, England: 1993) 12, 1551-1562 (2004).
9. Fradet-Turcotte, A. et al. 53BP1 is a reader of the DNA-damage-induced H2A Lys 15 ubiquitin mark. Nature 499, 50-54 (2013).
10. Mattiroli, F. et al. RNF168 ubiquitinates K13-15 on H2A/H2AX to drive DNA damage signaling. Cell 150, 1182-1195 (2012).
11. Canny, M.D. et al. Inhibition of 53BP1 favors homology-dependent DNA repair and increases CRISPR-Cas9 genome-editing efficiency. Nature Biotechnology 36, 95-102 (2018).
12. Dikic, I., Wakatsuki, S. & Walters, K. J. Ubiquitin-binding domains—from structures to functions. Nature Reviews. Molecular cell biology 10, 659-671 (2009).
13. Davis, L. & Maizels, N. Two Distinct Pathways Support Gene Correction by Single-Stranded Donors at DNA Nicks. Cell Reports 17, 1872-1881 (2016).
14. Verma, P. & Greenberg, R. A. Noncanonical views of homology-directed DNA repair. Genes & Development 30, 1138-1154 (2016).
15. Brault, J. et al. CRISPR-targeted MAGT1 insertion restores XMEN patient hematopoietic stem cells and lymphocytes. Blood 138, 2768-2780 (2021).
16. De Ravin, S. S. et al. Enhanced homology-directed repair for highly efficient gene editing in hematopoietic stem/progenitor cells. Blood 137, 2598-2608 (2021).
17. Sweeney, C. L. et al. Correction of X-CGD patient HSPCs by targeted CYBB cDNA insertion using CRISPR/Cas9 with 53BP1 inhibition for enhanced homology-directed repair. Gene Ther 28, 373-390 (2021).
18. Wienert, B. et al. Timed inhibition of CDC7 increases CRISPR-Cas9 mediated templated repair. Nat Commun 11, 2109 (2020).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of improving homology directed repair in a recipient cell, comprising:
expressing a Ubiquitin polypeptide variant corresponding to SEQ ID NO:1 in the recipient cell,
wherein an improvement of homology directed repair in the recipient cell is increased as compared to a cell that is not treated with the Ubiquitin polypeptide variant.

2. The method of claim 1, wherein the recipient cell is an immortalized cell.

3. The method of claim 2, wherein the immortalized cell is HEK293 kidney cell or Jurkat T cell.

4. The method of claim 1, wherein the recipient cell is a primary human T cell or an induced pluripotent stem cell.

5. The method of claim 1, wherein expressing the Ubiquitin polypeptide variant of SEQ ID NO:1 comprises transfecting SEQ ID NO:1 into the recipient cell.

6. The method of claim 1, wherein expressing the Ubiquitin polypeptide variant of SEQ ID NO:1 comprises transfecting SEQ ID NO:31 into the recipient cell, following by translation of SEQ ID NO:31 to produce SEQ ID NO:1.

7. A method of improving CRISPR-mediated gene editing in a target cell, comprising:
transfecting a Ubiquitin polypeptide variant corresponding to SEQ ID NO:1 or a Ubiquitin mRNA variant corresponding to SEQ ID NO:31 into the target cell that harbors a CRISPR ribonucleoprotein complex.

8. The method of claim 7, wherein the CRISPR ribonucleoprotein complex comprises a Cas9 polypeptide and a suitable guide RNA.

9. The method of claim 7, comprising transfecting a Ubiquitin polypeptide variant corresponding to SEQ ID NO:1 into the target cell that harbors a CRISPR ribonucleoprotein complex.

10. The method of claim 7, comprising transfecting a Ubiquitin mRNA variant corresponding to SEQ ID NO:31 into the target cell that harbors a CRISPR ribonucleoprotein complex.


SEQUENCE LISTING (31 sequences)

SEQ ID NO: 1 - protein, 74 aa, synthetic construct
MLIFVRMLTGKMIELEVEPSDTIENVKAKIQDHEGIPPDQQRLAFQGKSLEDGRTLSDYNILKDPKKMPLLRLR

SEQ ID NO: 2 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 3 - DNA, 20 bp, synthetic construct
  source 1..20: mol_type = other DNA
cagtcgcttcctgcctcgga

SEQ ID NO: 4 - DNA, 20 bp, synthetic construct
  source 1..20: mol_type = other DNA
aattatggggattactagga

SEQ ID NO: 5 - DNA, 20 bp, synthetic construct
  source 1..20: mol_type = other DNA
caaagtcctttcatctgtaa

SEQ ID NO: 6 - DNA, 20 bp, synthetic construct
  source 1..20: mol_type = other DNA
acctctggaaaaaggtaaga

SEQ ID NO: 7 - DNA, 20 bp, synthetic construct
  source 1..20: mol_type = other DNA
cttgccccacagggcagtaa

SEQ ID NO: 8 - DNA, 113 bp, synthetic construct
  source 1..113: mol_type = other DNA
accctcctctcttctttctgcagtcgcttcctgcctcggatacccatacgatgtgcctgattacgcaggatcacggctcagctggtatgacccaggtaaggaagagccacatg

SEQ ID NO: 9 - DNA, 86 bp, synthetic construct
  source 1..86: mol_type = other DNA
aaagactatgaaatggagagctaaattatggggattactagaattcggaaggggcagcaatgagttgacactacagacaaggcact

SEQ ID NO: 10 - DNA, 86 bp, synthetic construct
  source 1..86: mol_type = other DNA
tgtgtggtgagcgccctgggagccaaagtcctttcatctggaattctaaaggaccggttcatcaacttctttgtaggcaataccat

SEQ ID NO: 11 - DNA, 86 bp, synthetic construct
  source 1..86: mol_type = other DNA
attccaatgtgataggaactgtaacctctggaaaaaggtagaattcagaggggtgagctttccccttgcctgcccctactgggttt

SEQ ID NO: 12 - DNA, 200 bp, synthetic construct
  source 1..200: mol_type = other DNA
cagagccatctattgcttacatttgcttctgacacaactgtgttcactagcaacctcaaacagacaccatggtgcacctgactcctgtggaaaaatcgaattccgcagtcactgccctgtggggcaaggtgaacgtggatgaagttggtggtgaggccctgggcaggttggtatcaaggttacaagacaggtttaaggag

SEQ ID NO: 13 - DNA, 27 bp, synthetic construct
  source 1..27: mol_type = other DNA
aagaatgttgtgataaaaggtgatgct

SEQ ID NO: 14 - DNA, 24 bp, synthetic construct
  source 1..24: mol_type = other DNA
acacatccatgggacttctgcctc

SEQ ID NO: 15 - DNA, 23 bp, synthetic construct
  source 1..23: mol_type = other DNA
ccataccattcctctccacacaa

SEQ ID NO: 16 - DNA, 25 bp, synthetic construct
  source 1..25: mol_type = other DNA
gggaactgatgtgacttaccctatt

SEQ ID NO: 17 - DNA, 22 bp, synthetic construct
  source 1..22: mol_type = other DNA
cttgtccctctttgccttctct

SEQ ID NO: 18 - DNA, 22 bp, synthetic construct
  source 1..22: mol_type = other DNA
gggtggatctgagtggaagaaa

SEQ ID NO: 19 - DNA, 25 bp, synthetic construct
  source 1..25: mol_type = other DNA
agatatatcttagagggagggctga

SEQ ID NO: 20 - DNA, 25 bp, synthetic construct
  source 1..25: mol_type = other DNA
gcagagatattgctattgccttaac

SEQ ID NO: 21 - DNA, 60 bp, synthetic construct
  source 1..60: mol_type = other DNA
  misc_difference 55: note = RNA moiety
  misc_difference 60: note = 3'-terminal 3SpC3 moiety
acactctttccctacacgacgctcttccgatcttcagaactgtccttcaggttccaggtc

SEQ ID NO: 22 - DNA, 62 bp, synthetic construct
  source 1..62: mol_type = other DNA
  misc_difference 57: note = RNA moiety
  misc_difference 62: note = 3'-terminal 3SpC3 moiety
gtgactggagttcagacgtgtgctcttccgatctcagggcattacaaaagagaagactgacc

SEQ ID NO: 23 - DNA, 61 bp, synthetic construct
  source 1..61: mol_type = other DNA
  misc_difference 56: note = RNA moiety
  misc_difference 61: note = 3'-terminal 3SpC3 moiety
acactctttccctacacgacgctcttccgatctctgcatattctccccacagatagaagac

SEQ ID NO: 24 - DNA, 63 bp, synthetic construct
  source 1..63: mol_type = other DNA
  misc_difference 63: note = 3'-terminal 3SpC3 moiety
  misc_difference 58: note = RNA moiety
gtgactggagttcagacgtgtgctcttccgatctcctttagccttctcactgatatcgaatgg

SEQ ID NO: 25 - DNA, 60 bp, synthetic construct
  source 1..60: mol_type = other DNA
  misc_difference 60: note = 3'-terminal 3SpC3 moiety
  misc_difference 55: note = RNA moiety
acactctttccctacacgacgctcttccgatctctggtcttctcaaaggtgttggaggtg

SEQ ID NO: 26 - DNA, 61 bp, synthetic construct
  source 1..61: mol_type = other DNA
  misc_difference 61: note = 3'-terminal 3SpC3 moiety
  misc_difference 56: note = 3'-terminal 3SpC3 moiety
gtgactggagttcagacgtgtgctcttccgatctttgcctcagatcacactatctccacta

SEQ ID NO: 27 - DNA, 53 bp, synthetic construct
  source 1..53: mol_type = other DNA
acactctttccctacacgacgctcttccgatctcatgcccagtttctattggt

SEQ ID NO: 28 - DNA, 54 bp, synthetic construct
  source 1..54: mol_type = other DNA
gtgactggagttcagacgtgtgctcttccgatctctagcaacctcaaacagaca

SEQ ID NO: 29 - DNA, 54 bp, synthetic construct
  source 1..54: mol_type = other DNA
acactctttccctacacgacgctcttccgatcttcagaactgtccttcaggttc

SEQ ID NO: 30 - DNA, 56 bp, synthetic construct
  source 1..56: mol_type = other DNA
gtgactggagttcagacgtgtgctcttccgatctcagggcattacaaaagagaaga

SEQ ID NO: 31 - RNA, 425 nt, synthetic construct
  source 1..425: mol_type = other RNA
gggagacccaagctggctagcgtttaaacgggccctctagactcgagcggccgccaccatgctgatcttcgtgagaatgctgaccggcaagatgatcgaactggaagtggaacccagcgacaccatcgagaacgtgaaggccaaaatccaggaccacgagggcatccctcctgaccagcagagactggcctttcagggaaagtccctggaagatggaagaaccctgagcgactacaacatcctgaaggaccctaagaagatgccactgctgagactgagatgatcagcctcgactgtgccttctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa